(19) United States
(12) Patent Application Publication
(10) Pub. No.: US 2011/0002820 A1
(43) Pub. Date: Jan. 6, 2011

(54) EXTERIOR SURFACE TREATMENT SYSTEM

(76) Inventors: Howard Bernard Dawson, Derby (GB); Martin Balderstone, Derby (GB); Paul Peter Shanahan, Derby (GB)

Correspondence Address: HARRINGTON & SMITH, 4 RESEARCH DRIVE, Suite 202, SHELTON, CT, 06484-6212, US

(21) Appl. No.: 12/735,790

(22) PCT Filed: Feb. 20, 2009
(86) PCT No.: PCT/GB2009/000473

§ 371 Date: Sep. 13, 2010

(30) Foreign Application Priority Data
Feb. 20, 2008 (GB) 0803026.4

(51) Int. Cl. A61L 2/18; A01N 33/12; A01P 1/00

(52) U.S. Cl. 422/292; 514/643; 514/642

(57) ABSTRACT

An exterior surface treatment system for treating surfaces in the open air subject to weathering includes a container. The container is formed of a water soluble material and defines an interior. The system includes a composition located in the interior, the composition including one or more biocides, the total proportion of the biocide or biocides in the composition being greater than 40% w/w.

The present invention relates to exterior surface treatment systems for treating surfaces in the open air subject to weathering, particularly but not exclusively such systems for treating hard exterior surfaces.

The removal of soiling such as moss, algae, lichen, moulds and other biological growths from exterior surfaces such as patios, decking, masonry, garden walls, carport roofs, conservatory glass, garden fencing and roofing tiles is a widespread problem. Conventional treatments typically use a combination of corrosive chemicals such as hydrochloric acid, potassium hydroxide, sodium hypochlorite etc and/or high pressure spraying.

The use of such corrosive chemicals presents a number of major drawbacks. These chemicals lack any substantivity and if it rains following application the treatment is then negated. The corrosive nature of the product poses a substantial and real threat to untrained personnel using these products, and can cause significant damage to the treated surface which can affect any decorative appearance and will enhance the rate of re-soiling. Whilst these chemicals are very effective in removing inorganic salts from brickwork the level of effectiveness against biological growth is limited and often significant levels of mechanical work such as scrubbing, brushing etc are still required. These chemicals do not exhibit residual activity and will therefore not prevent re-growth of biological soil types.

The use of high pressure spraying techniques, either as a “stand alone” treatment or in combination with chemical treatments, also presents serious drawbacks. The process requires expensive equipment along with considerable time and effort. The high pressure spray can damage the surface layer of the substrate causing deterioration in decorative appearance over time and resulting in much more rapid re-soiling of the surface.

This damage is further exacerbated when high pressure spraying is used in combination with chemical treatments. High pressure spraying can result in removal of critical jointing material. The process requires large volumes of water to be used and often significant levels of mechanical [brushing or scrubbing] work are still required. There is no residual effect and given the reduction in re-soiling times the time between treatments becomes steadily shorter.

In this specification, all proportions are given by weight w/w, and are on a dry weight basis. In this specification, the term “exterior surface” is used to mean a surface which is in the open air, subject to weathering, as opposed to an “interior surface” which is located within a building or otherwise protected from weathering. An “emollient surfactant” is a surfactant which as well as being assessed as being non corrosive and non irritating to human skin and eyes and having no dermal toxicity, also has skin softening properties. Such surfactants are typically used in cosmetic and pharmaceutical applications.

According to a first aspect of the present invention, there is provided an exterior surface treatment system for treating surfaces in the open air subject to weathering, the system including a container, the container being formed of a water soluble material, the container defining an interior, the system including a composition located in the interior, the composition including one or more biocides, the total proportion of the biocide or biocides in the composition being greater than 40% w/w.

Possibly, the composition includes water, which may be present in a proportion no greater than 20% w/w of the total composition, and possibly no less than 10% w/w of the total composition.

Possibly the total proportion of the biocide or biocides in the composition is at most 80% w/w.

Possibly, the or each biocide is a surfactant, and may be a cationic surfactant, and may be a membrane active biocide.

Possibly, the or each biocide is one or more selected from a group consisting of: quaternary ammonium compounds, amphoterics, amphoquats, alkylamines, biguanides and imidazolium derivatives.

Possibly, the or each quaternary ammonium biocide is selected from benzalkonium chloride and didecyl dimethyl ammonium chloride.

Possibly, the or each amphoteric biocide is selected from alkylpropylene diamine glycine and alkylpropylene triamineglycine.

Possibly, the amphoquat biocide is a cocos ammoniumbetaine type.

Possibly, the alkylamine biocide is selected from alkylpropylene diamine and alkylpropylene triamine.

Possibly, the biguanide biocide is polyhexamethylene biguanide.

Possibly, the imidazolium derivative biocide is 1,3-dialkyl-2-methyl imidazoliumchloride.

Possibly the composition includes a non ionic surfactant, and may include a plurality of non ionic surfactants. Possibly the total proportion of the non ionic surfactant or surfactants in the composition is at most 50% w/w, and may be at least 5% w/w

Possibly, the or each non ionic surfactant is a non classified material for skin and eye irritancy and dermal toxicity. Possibly, the or each non ionic surfactant is one or more selected from a group consisting of: EO-PO block copolymers, castor oil ethoxylates, amine oxides, fatty acid triglycerides, amine ethoxylates and fatty acid ethoxylates. Possibly, the or each non ionic surfactant does not include an alcohol ethoxylate.

Possibly, the or each non ionic surfactant is an emollient surfactant, and may be selected from a group consisting of a castor oil ethoxylate, a fatty acid triglyceride and a coconut amine ethoxylate.

Possibly, the water soluble material is a flexible material, and may be a film material, and may be formed of poly vinyl alcohols or poly vinyl acetates.

Possibly, the composition includes one or more of the group consisting of water repellents, herbicides, and sequestering agents.

Possibly, the composition is non corrosive to human skin. Possibly, the non corrosive composition includes a biocide and an emollient non ionic surfactant.

Possibly, the composition does not include an organic solvent, which may comprise an alcohol, a glycol and/or an ether.

Possibly, the composition comprises a surfactant system, which may only comprise the one or more biocidal surfactants and the one or more non ionic surfactants. Possibly, the surfactant system does not include an anionic surfactant.

Possibly, the composition includes citric acid, and may include citric acid at between 2 and 5% w/w.

Possibly, the ratio of the weight proportion of biocides to non ionic surfactants in the composition is at least 0.8:1. Possibly, in use, the container is added to water to form a diluted composition, which may have a resultant total proportion of biocide or biocides of up to 0.6% w/w. Possibly, the resultant total proportion of biocide or biocides is at least 0.3% w/w.

Possibly, in use the exterior treatment system is for the removal of mosses, algae, lichen, moulds and other forms of organic growth from exterior hard surfaces.

According to a second aspect of the present invention, there is provided a non corrosive biocidal composition, the composition including one or more biocides and an emollient non ionic surfactant, the composition being non corrosive to human skin, the total proportion of the biocide or biocides in the composition being greater than 40% w/w.

The total proportion of the biocide in the composition may be up to 65% w/w.

Possibly, the biocide of the non corrosive composition is a quaternary ammonium compound, and may be selected from benzalkonium chloride and didecyl dimethyl ammonium chloride. Possibly, the composition comprises at least 20% w/w of the emollient non ionic surfactant, and may comprise up to 50% w/w of the emollient non ionic surfactant, which may be selected from a group containing a castor oil ethoxylate, a fatty acid triglyceride and a coconut amine ethoxylate.

Possibly, the ratio of the weight proportion of biocides to non ionic surfactants in the non corrosive biocidal composition is up to 3:1, and may be at least 0.8:1.

The non corrosive biocidal composition may be for treating surfaces in the open air subject to weathering, and may form part of an external surface treatment system, and may include any of the features described in the preceding paragraphs.

According to a third aspect of the present invention, there is provided a method of treatment of an exterior surface, the method including the use of a exterior surface treatment system, the system including a container, the container being formed of a water soluble material, the container defining an interior, the system including a composition located in the interior, the composition including one or more biocides, the total proportion of the biocide or biocides in the composition being greater than 40% w/w.

Possibly, the exterior treatment system includes any of the features described in the preceding statements.

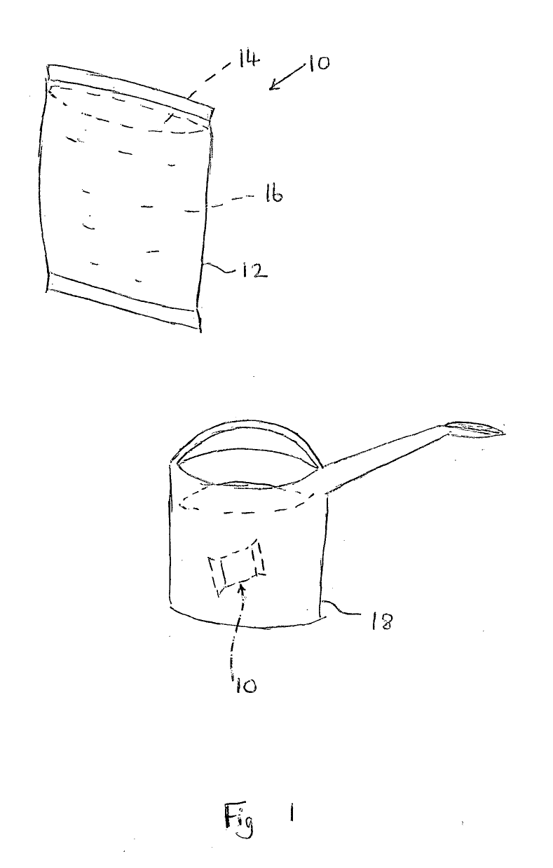

Embodiments of the present invention will now be described, by way of example only, and with reference to FIG. 1, which is a schematic diagram showing steps in the use of an exterior surface treatment system.

Referring to FIG. 1, an exterior surface treatment system 10 for treating surfaces in the open air subject to weathering includes a container in the form of a sachet 12, which is formed of a flexible water soluble film material, which could be formed of polyvinyl alcohols or polyvinyl acetates. The sachet 12 defines an interior 14. The system 10 includes a composition 16 located in the interior 14. The composition 16 substantially fills the interior 14. The sachet 12 is sealed following filling.

Table 1 gives parameters for the composition 16. The composition 16 includes a biocide and could include a plurality of biocides. The total proportion of the biocide or biocides in the composition is greater than 40% w/w, and is at most 80% w/w (proportions are on a dry weight basis).

TABLE 1
Composition parameters

| Ingredient | Ingredient proportion % w/w |
|---|---|
| Biocide | Greater than 40%, at most 80% |
| Non ionic surfactant | 0%-50% |
| Water | 10%-20% |
| Other | 0%-5% |

The biocide could be a membrane active cationic surfactant biocide. Examples of suitable such biocides are quaternary ammonium compounds such as benzalkonium chloride and didecyl dimethyl ammonium chloride, amphoterics such as alkylpropylene diamine glycine and alkylpropylene triamineglycine, amphoquats such as cocos ammoniumbetaine types, alkylamines such as alkylpropylene diamine and alkylpropylene triamine, biguanides such as polyhexamethylene biguanide and imidazolium derivatives such as 1,3-dialkyl-2-methyl imidazoliumchloride.

The composition 16 optimally could include a non-ionic surfactant, and could include a plurality of non-ionic surfactants. The total proportion of the non-ionic surfactant or surfactants in the composition could be at most 50% w/w, and could, optimally be at least 5% w/w. The ratio of biocide to non ionic surfactant is at least 0.8:1.

The or each non-ionic surfactant could optimally be a non classified material for skin and eye irritancy and dermal toxicity, and could be selected from a group consisting of EO-PO block copolymers, castor oil ethoxylates, amine oxides, fatty acid triglycerides, amine ethoxylates and fatty acid ethoxylates.

For reasons of skin and eye irritancy and dermal toxicity, alcohol ethoxylate non ionic surfactants such as the Neodol® range of non ionic surfactants are specifically not included in the compositions of the invention. The inventors have also surprisingly found that the required functionality can be obtained in the compositions of the invention without those compositions including any organic solvents, such as alcohol, glycol, and/or ether.

The composition thus comprises a surfactant system which comprises only the one or more cationic surfactant biocides and the one or more non ionic surfactants. The surfactant system of the composition specifically does not include an anionic surfactant, as this would be liable to react with the cationic surfactant.

The composition 16 includes water in a proportion of no less than 10% w/w of the total composition, and no greater than 20% w/w of the total composition. An amount of water is required in the composition 18 for the sachet 16 to retain flexibility, but the amount of water should be limited as otherwise the sachet 16 will begin to dissolve.

The composition 16 could include other ingredients such as water repellents, citric acid, herbicides and sequestering agents, which could be in the range 0-5% w/w.

In use, the composition 16 is prepared and placed into the interior 14 of the water soluble sachet 12. The sachet 16 is added to water within a container 18, which could, for example, be a watering can. In the water, the sachet 16 dissolves, releasing the composition 16 which in turn dissolves. Thus the sachet 16 does not need to be cut or torn to release the composition 16. In one example, the sachet 12 contains approximately 37.5 g of the composition, which is added to between 2.5 and 10 litres of water, and in one example could be added to 5 litres of water. Thus from the parameters of Table 1, the proportion of biocide in the diluted composition is between 0.15% w/w and 1.2% w/w and optimally is between 0.3% w/w and 0.6% w/w.

Table 2, found at the end of this description, shows more specific examples of possible compositions, which are discussed below. All of the example compositions include a quaternary ammonium biocide, either benzalkonium chloride (BAC) or didecyl dimethyl ammonium chloride (DDAC) or a combination thereof. These compounds are typically supplied as 80% concentrates with water. The quantities shown in the table are shown as dry weight quantities, with the water shown as a separate ingredient. Quaternary ammonium compounds are less aggressive than conventional chemicals used in the treatment of exterior surfaces and are less likely to cause damage to decorative surfaces. They pose less risk to users handling the products. They have a good level of activity against a wide range of organic soiling types and they give a degree of residual activity.

In examples 1, 2 and 3, the sachet 12 is filled with quaternary ammonium concentrate to form the exterior treatment system 10. However, as shown in Table 3 these compounds solidify if stored below 5° C. and have poor solubility in water (also shown in Table 3) requiring a considerable level of agitation, particularly when the diluent water is at temperatures below 10° C. For these reasons, compositions only comprising BAC and/or DDAC are considered less suitable for use in sachets of a exterior surface treatment system 10.

Other biocides could be used instead of, or in addition to, BAC or DDAC. In example 9, Tegotain® AFB, a blend of amphoteric alkylaminocarboxylate with alkylbetaine (an amphoquat) is included in addition to BAC. Tegotain® AFB is supplied at 100% concentration. In example 10, polyhexamethylene biguanide is used in addition to BAC and DDAC.

Composition examples 4-14 each include a non-ionic surfactant or surfactants. Examples 4-10 include different combinations of suitable types of non ionic surfactants, each of which is a non classified material for skin and eye irritancy and dermal toxicity. Suitable types of non ionic surfactants used in the example compositions are EO-PO block copolymers (suitable examples of which are marketed under the trade names Pluronic® PE3100, Pluronic® PE8100, and Pluronic® PE6200), fatty acid triglycerides (one suitable example of which is marketed under the trade name Surfac® MCTG), castor oil ethoxylates (one suitable example of which is marketed under the trade name Emulan® EL40), and coconut amine ethoxylates (one suitable example of which is marketed under the trade name Ethomeen® C/15).

The proportion of total non-ionic surfactants for examples 4-10 varies from 6% w/w for example 4, to 12% w/w for examples 5-9, with 10% w/w for example 10.

TABLE 3

| | | Dissolution Time | | | | |
|---|---|---|---|---|---|---|
| | Solidification | [mins] v Temp. [C.] | | | | |
| Example | Temp [C.] | 25 C. | 15 C. | 10 C. | 5 C. | 2 C. |
| 1 | >5 | 1.0 | 2.5 | 5.0 | 10.0 | >20.0 |
| 2 | >5 | 1.5 | 3.0 | 5.5 | 10.0 | >20.0 |
| 3 | >5 | 1.5 | 3.0 | 5.0 | 10.0 | >20.0 |
| 4 | 2 | 0.5 | 1.5 | 3.0 | 5.0 | >10.0 |
| 5 | >0 | 0.2 | 0.8 | 2.0 | 3.0 | >5.0 |
| 7 | >0 | 0.2 | 0.5 | 0.8 | 1.0 | >20.0 |
| 10 | >0 | 0.2 | 0.3 | 0.5 | −0.8 | 1.0 |

The effect of the non-ionic surfactant on the solidification temperature and dissolution time in water [1% formulation] at various temperatures is shown in Table 3.

For example compositions 1, 2 and 3 without non-ionic surfactants, the solidification temperature is greater than 5° C. For example composition 4, with 6% w/w non-ionic surfactants, the solidification temperature is reduced to 2° C. and the dissolution times are reduced relative to those for example compositions 1, 2 and 3. For example compositions 5, 7 and 10, with total non-ionic surfactants of 10 or 12% w/w, the solidification temperatures are less than 0° C., and the dissolution times have again reduced relative to example 4 and example compositions 1-3.

The data shown in Table 3 clearly demonstrates the benefits of the addition of the non-ionic surfactant or surfactants to the biocide. Indeed without these additional types of surfactant the products would have very limited practical use in this type of system.

Thus, the use of non-ionic surfactants reduces the solidification temperature and increases the speed of dissolution of the composition, improving the usability of the composition.

The algaecidal activity of BAC and DDAC are shown in Table 4:

TABLE 4

| | Algistatic concentration [ppm] against various algae | | | |
|---|---|---|---|---|
| | Anabaena | Chlorella | Oscillatoria | Stiegeocionim |
| Biocide | cylindrica | vulgaris | tenius | sp. |
| BAC | 1.0 | 1.0 | 0.6 | 0.7 |
| DDAC | 0.2 | 2.0 | 0.7 | 0.7 |

The fungicidal activity of BAC and DDAC are shown in Table 5:

TABLE 5
Fungicidal activity of BAC and DDAC

| | Minimum inhibitory concentration [ppm] against fungi | | | |
|---|---|---|---|---|
| | Chaetomium | Myrothecium | Trichoderma | Aspergillus |
| Biocide | globosum | verrucaria | viridae | niger |
| BAC | 7 | 150 | 75 | 75 |
| DDAC | 7 | 20 | 20 | 75 |

In use, the composition 16 is prepared, and placed into the interior 14 of the water soluble sachet 12 as described previously and the diluted composition is applied at a rate of approximately 1 litre per 3-5 square metres onto an exterior surface to be cleaned.

In one example, the diluted composition was applied to a variety of surfaces, for example concrete paving stones, brick, wooden decking, block paving, plastic roofing, glass, sandstone masonry work etc., which were coated with a variety of algae, lichen, mosses and other forms of biological soiling. No mechanical work such as brushing or scrubbing was carried out, nor were the surfaces treated with high pressure water spray.

Following treatment, the treated surfaces were left untouched. Examination after various time periods [1, 2, 3, 5 and 7 days] following treatment clearly showed the effectiveness of the various biocidal treatments, with the biological soiling being removed or significantly reduced. Moreover, examination after longer periods [1-6 months] following treatment clearly demonstrated, the residual effectiveness of the treatments. The “residual effectiveness” is an indication that the composition is not simply washed away when the treated exterior surface is subsequently subject to weathering, and is an important functional characteristic of such exterior treatment compositions, as it indicates that some residual biocide remains on the exterior surface, inhibiting organic soiling.

Although example compositions 1-10 as described above are relatively less aggressive than conventional compositions typically used in exterior surface treatment applications, they are still classified as corrosive to human skin when tested in accordance with regulatory requirements, and therefore have to be packaged and labelled as corrosive substances. While undertaking research, the applicants were surprised to discover that certain biocides in the form of quaternary ammonium compounds, when combined with a non-ionic emollient surfactant, produced compositions which were assessed to be non corrosive to human skin when tested in accordance with regulatory requirements.

In one example, the quaternary ammonium compounds could be selected from benzalkonium chloride and didecyl dimethyl ammonium chloride. The composition could include greater than 40% w/w of the quaternary ammonium compound, and could include the quaternary ammonium compound up to 65% w/w. The composition could comprise at least 20% w/w of the emollient non ionic surfactant, and could comprise up to 50% w/w of the emollient non ionic surfactant, which could be selected from a group containing a castor oil ethoxylate, a fatty acid triglyceride, and a coconut amine ethoxylate.

The ratio of the weight proportion of biocides to non ionic surfactants in the non corrosive biocidal composition could be up to 3:1, and could be at least 0.8:1.

In Table 2, example compositions 11 to 13 are examples of non corrosive compositions. These compositions were tested using the Episkin™ in vitro Reconstituted Human Epidermis Model after treatment periods of 3, 60 and 240 minutes to meet the requirements of the OECD Guidelines for the Testing of Chemicals No 431 “In Vitro Skin Corrosion: Human Skin Model Test”. When tested in accordance with these guidelines, these compositions were found to be non corrosive, and therefore do not require packaging or labelling as corrosive materials.

In these compositions, the use of the non-ionic emollient surfactant at additional levels of at least 20% w/w was found to reduce the solidification point of the composition, improve the dissolution characteristics and boost the cleaning effect.

In use, the non corrosive biocidal composition can be prepared and placed into the interior 14 of the water-soluble sachet 12 as previously described, to permit ease of handling and dosing, and the sachet 12 added to a quantity of water in a container 18 again as previously described.

Example 14 is a further example of a non corrosive composition, which is a modification of example 11, in which a proportion of the biocide of example 11 has been replaced with citric acid. The citric acid has been found to be effective in neutralizing manganese hydroxide, which produces a dark stain in masonry such as limestone and Portland stone. In this formulation, the citric acid is not just acting as a pH modifier, but as a neutralizing agent for the manganese hydroxide. The citric acid could be added in proportions in the range between 2 and 5% w/w.

Various other modifications could be made without departing from the scope of the invention. The container could be of any form, and could be formed of any suitable water soluble material, and could be of any suitable size and shape, and contain any suitable amount of the composition.

Any feature of any of the compositions described could be combined in any suitable way.

There is thus provided an exterior surface treatment system including a composition which is stable at low temperatures, dissolves readily with a minimum of agitation when diluted at a range of temperatures down to 2° C., exhibits high biological efficacy against a wide range of mosses, algae, lichen, moulds and other biological soils attached to a variety of exterior hard surfaces when applied either as a drench (e.g. by a watering can or a coarse low pressure spray), and imparts significant long term residual protection to treated surfaces preventing regrowth for periods up to six months following treatment. The composition is stored in water soluble sachets which can be easily added to containers. The composition is stored and transported in concentrated form, minimizing storage and transportation costs. Dosing is simple, and the possibility of contact between the composition and the user is minimized. Should any contact occur, the ingredients have been specifically chosen to reduce the hazard, and the invention further provides non corrosive biocidal compositions, which have been assessed as being non corrosive in contact with human skin.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

TABLE 2
Example compositions

| | Example of | | | | | | | | |
|---|---|---|---|---|---|---|---|---|---|
| | ingredient trade | | Example | | | | | | |
| Ingredient | name | | 1 | 2 | 3 | 4 | 5 | 6 | 7 |
| Biocides | | | | | | | | | |
| Benzalkonium chloride (BAC) | | % w/w | 80 | | 40 | 75.2 | 70.4 | 35.2 | 70.4 |
| Didecyl dimethyl ammonium | | % w/w | | 80 | 40 | | | 35.2 | |
| chloride (DDAC) | | | | | | | | | |
| Amphoteric | Tegotain ® AFB | % w/w | | | | | | | |
| alkylaminocarboxylate with | | | | | | | | | |
| alkylbetaine | | | | | | | | | |
| Polyhexamethylene biguanide | | % w/w | | | | | | | |
| Sub total biocides | | % w/w | 80 | 80 | 80 | 75.2 | 70.4 | 70.4 | 70.4 |
| Non ionic surfactants | | | | | | | | | |
| EO-PO block copolymer | Pluronic ® PE3100 | % w/w | | | | | | | 4 |
| EO-PO block copolymer | Pluronic ® PE8100 | % w/w | | | | | | | |
| EO-PO block copolymer | Pluronic ® PE6200 | % w/w | | | | 3 | 3 | | |
| Fatty acid triglyceride | Surfac ® MCTG | % w/w | | | | 3 | 3 | 6 | 8 |
| Castor oil ethoxylate | Emulan ® EL40 | % w/w | | | | | 6 | 6 | |
| Coconut amine ethoxylate | Ethomeen ® C/15 | % w/w | | | | | | | |
| Sub total non ionic | | % w/w | | | | 6 | 12 | 12 | 12 |
| surfactants | | | | | | | | | |
| Ratio of biocides:non ionic | | | | | | 12.53 | 5.87 | 5.87 | 5.87 |
| surfactants | | | | | | | | | |
| Citric Acid | | % w/w | | | | | | | |
| Water | | % w/w | 20 | 20 | 20 | 18.8 | 17.6 | 17.6 | 17.6 |
| Total | | % w/w | 100 | 100 | 100 | 100 | 100 | 100 | 100 |

| | | | | | | | | |
|---|---|---|---|---|---|---|---|---|
| Proportion of biocide in diluted composition at | % w/w | 0.596 | 0.596 | 0.596 | 0.560 | 0.524 | 0.524 | 0.524 |
| point of use, based on 37.5 g composition in 5 l | | | | | | | | |
| water | | | | | | | | |

| | Example of | | | | | | | |
|---|---|---|---|---|---|---|---|---|
| | ingredient trade | Example | | | | | | |
| Ingredient | name | 8 | 9 | 10 | 11 | 12 | 13 | 14 |
| Biocides | | | | | | | | |
| Benzalkonium chloride (BAC) | | 35.2 | 48 | 32 | 62.5 | 50 | 41.7 | 57.5 |
| Didecyl dimethyl ammonium | | 35.2 | | 32 | | | | |
| chloride (DDAC) | | | | | | | | |
| Amphoteric | Tegotain ® AFB | | 28 | | | | | |
| alkylaminocarboxylate with | | | | | | | | |
| alkylbetaine | | | | | | | | |
| Polyhexamethylene biguanide | | | | 10 | | | | |
| Sub total biocides | | 70.4 | 76 | 74 | 62.5 | 50 | 41.7 | 57.5 |
| Non ionic surfactants | | | | | | | | |
| EO-PO block copolymer | Pluronic ® PE3100 | | | | | | | |
| EO-PO block copolymer | Pluronic ® PE8100 | | 4 | | | | | |
| EO-PO block copolymer | Pluronic ® PE6200 | | | 4 | | | | |
| Fatty acid triglyceride | Surfac ® MCTG | 6 | | | | | | |
| Castor oil ethoxylate | Emulan ® EL40 | 6 | 8 | | 21.9 | 37.5 | 47.9 | 21.9 |
| Coconut amine ethoxylate | Ethomeen ® C/15 | | | 6 | | | | |
| Sub total non ionic | | 12 | 12 | 10 | 21.9 | 37.5 | 47.9 | 21.9 |
| surfactants | | | | | | | | |
| Ratio of biocides:nonionic | | 5.87 | 6.33 | 7.40 | 2.85 | 1.33 | 0.87 | 2.63 |
| surfactants | | | | | | | | |
| Citric Acid | | | | | | | | 5.0 |
| Water | | 17.6 | 12 | 16 | 15.6 | 12.5 | 10.4 | 15.6 |
| Total | | 100 | 100 | 100 | 100 | 100 | 100 | 100 |

| | | | | | | | |
|---|---|---|---|---|---|---|---|
| Proportion of biocide in diluted composition at | 0.524 | 0.566 | 0.551 | 0.465 | 0.372 | 0.310 | 0.428 |
| point of use, based on 37.5 g composition in 5 l | | | | | | | |
| water | | | | | | | |

1. An exterior surface treatment system for treating surfaces in the open air subject to weathering, the system including a container, the container being formed of a water soluble material, the container defining an interior, the system including a composition located in the interior, the composition including one or more biocides, the total proportion of the biocide or biocides in the composition being greater than 40% w/w, the composition also including a non ionic emollient surfactant.

2. A treatment system according to claim 1, in which the composition includes water.

3. A treatment system according to claim 2, in which the water is present in a proportion no greater than 20% w/w of the total composition.

4. (canceled)

5. (canceled)

6. A treatment system according to claim 1, in which the or each biocide is a surfactant.

7. A treatment system according to claim 6, in which the or each biocide is a membrane active biocide.

8. A treatment system according to claim 1, in which the or each biocide is one or more selected from a group consisting of: quaternary ammonium compounds, amphoterics, amphoquats, alkylamines, biguanides and imidazolium derivatives.

9. A treatment system according to claim 8, in which the or each quaternary ammonium biocide is one or more selected from benzalkonium chloride and didecyl dimethyl ammonium chloride.

10. (canceled)

11. (canceled)

12. (canceled)

13. A treatment system according to claim 8, in which the biguanide biocide is polyhexamethylene biguanide.

14. (canceled)

15. (canceled)

16. (canceled)

17. A treatment system according to claim 1, in which the total proportion of the non ionic surfactant or surfactants in the composition is at most 50% w/w.

18. (canceled)

19. (canceled)

20. (canceled)

21. (canceled)

22. A treatment system according to claim 1, in which the emollient non ionic surfactant is one or more selected from a group consisting of a castor oil ethoxylate, a fatty acid triglyceride and a coconut amine ethoxylate.

23. (canceled)

24. (canceled)

25. A treatment system according to claim 1, in which the water soluble material is formed of poly vinyl alcohols or poly vinyl acetates.

26. (canceled)

27. (canceled)

28. (canceled)

29. (canceled)

30. (canceled)

31. (canceled)

32. (canceled)

33. A treatment system according to claim 1, in which the composition includes citric acid.

34. A treatment system according to claim 33, in which the composition includes citric acid at between 2 and 5% w/w.

35. A treatment system according to claim 1, in which the ratio of the weight proportion of biocides to non ionic surfactants in the composition is at least 0.8:1.

36. (canceled)

37. (canceled)

38. (canceled)

39. (canceled)

40. A non corrosive biocidal composition, the composition including one or more biocides and an emollient non ionic surfactant, the composition being non corrosive to human skin, the total proportion of the biocide or biocides in the composition being greater than 40% w/w.

41. (canceled)

42. A non corrosive biocidal composition according to claim 40, in which the biocide of the non corrosive composition is a quaternary ammonium compound.

43. A non corrosive biocidal composition according to claim 42, in which the quaternary ammonium compound is one or both selected from benzalkonium chloride and didecyl dimethyl ammonium chloride.

44. A non corrosive biocidal composition according to claim 40, in which the composition comprises at least 20% w/w of the emollient non ionic surfactant.

45. (canceled)

46. A non corrosive biocidal composition according to claim 40, in which the emollient surfactant is one or more selected from a group containing a castor oil ethoxylate, a fatty acid triglyceride and a coconut amine ethoxylate.

47. (canceled)

48. A non corrosive biocidal composition according to claim 40, in which the ratio of the weight proportion of biocides to non ionic surfactants in the non corrosive biocidal composition is at least 0.8:1.

49. (canceled)

50. (canceled)

51. (canceled)

52. (canceled)

53. (canceled)

54. (canceled)

55. (canceled)

56. (canceled)

57. (canceled)
